(19) United States
(12) Patent Application Publication
(10) Pub. No.: US 2008/0213330 A1
(43) Pub. Date: Sep. 4, 2008

(54) Pharmaceutical Compositions Comprising Polyethylene Glycol Having a Molecular Weight of Less Than 600 Daltons

(76) Inventor: Olivier Lambert, Spechbach-le-Haut (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/570,061

(22) PCT Filed: Jun. 8, 2005
(86) PCT No.: PCT/EP2005/006173

§ 371 Date: Nov. 13, 2007

(30) Foreign Application Priority Data
Jun. 9, 2004 (GB) 0412866.6

(51) Int. Cl. A61F 13/00; A61K 38/00; A61K 31/70

(52) U.S. Cl. 424/422; 514/2; 514/12; 514/44

(57) ABSTRACT

A liquid pharmaceutical composition comprising a biodegradable polymer, polyethylene glycol having a molecular weight of less than 600 Daltons, a pharmaceutically active agent and less than 0.5% of an biologically acceptable organic solvent.

The present invention relates to liquid pharmaceutical compositions, in particular to depot formulations comprising a pharmaceutically active agent and to a process for preparing said depot formulations.

Depot formulations are typically administered parenterally. The active agent in liquid form may be administered by injection subcutaneously or intramuscularly through a small gauge needle or placed into accessible tissue sites through a cannula. However parenteral administration may be very painful especially if repeated injections are necessary. Furthermore, there may be difficulties with depot formulations which are administered in liquid form comprising more than 50% of an organic solvent and which form a solid implant in the body after injection. Often the solidifying process starts in the syringe before injection and causes needle clogging. Depot formulations which form implants after injection may comprise a polymer or a mixture of polymers. These polymers have to be dissolved in an organic solvent. If the organic solvent remains in the solution for injection it might cause severe tissue irritation or necrosis at the site of implantation.

A variety of approaches have been developed to provide processes for preparing depot formulations. However these processes are often very complex comprising many different steps. Several processes include a heating step which might cause severe stability problems, e.g. of the excipients of the depot formulation such as polymers.

There is a need to provide improved depot formulations and simplified processes to prepare said depot formulations to overcome the above mentioned difficulties.

Surprisingly it has been found that advantageous parenteral depot formulations with a biodegradable polymer may be obtained if the composition comprises polyethylene glycol (PEG) with a molecular weight of less than 600 Daltons and less than about 0.5% of any other organic solvent.

The present invention provides in one aspect a liquid composition comprising
(a) a biodegradable polymer,
(b) a polyethylene glycol having a molecular weight of less than 600 Daltons,
(c) a pharmaceutically active agent, and
(d) less than about 0.5% of a pharmaceutically acceptable solvent, and optionally
(e) an additive

The composition of the invention may be stored e.g. in prefilled syringe over an extended period of time without precipitation. Further, the compositions of the invention are well tolerated, e.g. may show only negligible irritating, necrotic or toxic effects.

The depot formulations of the present invention are adapted to release all or substantially all the active agent over an extended period of time.

In another aspect the invention provides a process for preparing a depot formulation comprising the steps:
(i) dissolving a biodegradable polymer in a pharmaceutically acceptable organic solvent
(ii) optionally adding an additive to the polymer/solvent solution of step (i)
(iii) dissolving a pharmaceutically active agent in polyethylene glycol with a molecular weight of less than 600 Daltons
(iv) mixing solution (i) and (iii) or (ii) and (iii)
(v) removing the pharmaceutically acceptable organic solvent from the mixture e.g. by stirring and N₂ urging under reduced pressure or by tangential cross-flow filtration to afford dia-filtration.

In a further aspect the invention provides a process for preparing a depot formulation comprising the steps:
(i) dissolving a biodegradable polymer in a pharmaceutically acceptable organic solvent
(ii) optionally mixing an additive with polyethylene glycol with a molecular weight of less than 600 Daltons.
(iii) dissolving a pharmaceutically active agent in polyethylene glycol with a molecular weight of less than 600 Daltons or in the polyethylene glycol/additive mixture of step (ii)
(iv) mixing solution (i) and (iii)
(v) removing the pharmaceutically acceptable organic solvent from the mixture e.g by stirring and N₂ urging under reduced pressure or by tangential cross-flow filtration to afford dia-filtration.

According to the invention, a pharmaceutically acceptable, organic solvent is used to dissolve the biodegradable polymer but this solvent is removed at the end of the process. The resulting compositions of the invention contain only minor amounts of organic solvent, e.g. irritating solvent, e.g. less than 0.5% by weight based on the total weight of the composition.

The polymer of the composition of the invention may be a synthetic or a natural polymer. The polymer may be either a biodegradable or non-biodegradable or a combination of biodegradable and non-biodegradable polymers, preferably a biodegradable polymer may be used.

By “polymer” is meant a homopolymer or a copolymer.

As used herein, “biodegradable” means a material that should degrade by bodily processes to products readily disposable by the body and should not accumulate in the body.

Suitable polymers include

(a) linear or branched polyesters which are linear chains radiating from a polyol moiety, e.g. glucose,

(b) polyesters such as D-, L- or racemic polylactic acid, polyglycolic acid, polyhydroxy-butyric acid, polycaprolactone, polyalkylene oxalate, polyalkylene glycol esters of acids of the Kreb's cycle, e.g. citric acid cycle, and the like and combinations thereof,

(c) polymers of organic ethers, anhydrides, amides, and orthoesters,

(d) copolymers of organic esters, ethers, anhydrides, amides, and orthoesters by themselves or in combination with other monomers.

The polymers may be cross-linked or non-cross-linked. Usually not more than 5%, typically less than 1% are cross-linked.

The preferred polymers of this invention are linear polyesters, and branched chain polyesters. The linear polyesters may be prepared from the α-hydroxy carboxylic acids, e.g. lactic acid and glycolic acid, by condensation of the lactone dimers, see e.g. U.S. Pat. No. 3,773,919, the contents of which are incorporated herein by reference. The preferred polyester chains in the linear or branched (star) polymers are copolymers of the c-carboxylic acid moieties, lactic acid and glycolic acid, or of the lactone dimers. The molar ratios of lactide: glycolide of polylactide-co-glycolides preferably used according to the invention is preferably from about 95:5 to 5:95, e.g. 75:25 to 25:75, e.g. 60:40 to 40:60, with from 55:45 to 45:55, e.g. 52:48 to 48:52, e.g. 50:50.

Linear polyesters, e.g. linear polylactide-co-glycolides (PLG), preferably used according to the invention have a weight average molecular weight (Mw) between about 1,000 and about 50,000 Da, e.g. about 10,000 Da, and a polydispersity M_{w}/Mₙ e.g. between 1.2 and 2. The intrinsic viscosities of linear polymers of Mw 1000 to 50,000 are 0.05 to 0.6 dl/g, in chloroform. Suitable examples include e.g. those commonly known and commercially available as Resomers® from Boehringer Ingelheim, in particular Resomers® RG, e.g. Resomer® RG 502, 502H, 503, 503H, 504, 504H.

Branched polyesters, e.g. branched polylactide-co-glycolides, preferably used according to the invention may be prepared using polyhydroxy compounds e.g. polyol e.g. glucose or mannitol as the initiator. These esters of a polyol are known and described e.g. in GB 2,145,422 B, the contents of which are incorporated herein by reference. The polyol contains at least 3 hydroxy groups and has a molecular weight of up to 20,000 Da, with at least 1, preferably at least 2, e.g. as a mean 3 of the hydroxy groups of the polyol being in the form of ester groups, which contain poly-lactide or co-poly-lactide chains. Typically 0.2% glucose is used to initiate polymerization. The branched polyesters (Glu-PLG) have a central glucose moiety having rays of linear polylactide chains, e.g. they have a star shaped structure.

The branched polyesters having a central glucose moiety having rays of linear polylactide-co-glycolide chains (Glu-PLG) may be prepared by reacting a polyol with a lactide and preferably also a glycolide at an elevated temperature in the presence of a catalyst, which makes a ring opening polymerization feasible.

The branched polyesters having a central glucose moiety having rays of linear polylactide-co-glycolide chains (Glu-PLG) preferably have a weight average molecular weight M_{w} in the range of from about 1,000 to 55,000, preferably 20,000, e.g. 10,000 Da, and a polydispersity e.g. of from 1.1 to 3.0, e.g. 2.0 to 2.5. The intrinsic viscosities of star polymers of Mw 10,000 to M_{w} 50,000 are 0.05 to 0.6 dl/g in chloroform. A star polymer having a M_{w} of 50,000 has a viscosity of 0.5 dl/g in chloroform.

The desired rate of degradation of polymers and the desired release profile for compounds of the invention may be varied depending on the kind of monomer, whether a homo- or a copolymer or whether a mixture of polymers is employed.

A mixture of polymers may comprise at least two different kinds of polymers, e.g. as listed under (a) to (e) above, or two polymers of the same polymer class with different properties. For example, a mixture of polymers may comprise a polymer having a medium weight average molecular weight, e.g. from about 30,000 to about 50,000 Da, e.g. of about 20,000 Da, and of a polymer having a low weight average molecular weight, e.g. of about 2.000 to about 20,000 Da, e.g. of about 10,000 Da.

Preferably, the polymer matrix comprises a linear and/or branched polylactide-co-glycolide. More preferably, the polymer matrix comprises a Resomer® RG and/or a star polylactide-co-glycolide polymer having a weight average molecular weight of about 10,000 Da and/or a star polylactide-co-glycolide polymer having a weight average molecular weight of about 50,000 Da. The ratio of linear to branched polylactide-co-glycolide preferably is 0:100 to 100:0, e.g. 50:50 to 25:75.

The solvent of the present invention may be miscible with polyethylene glycol. Examples of such solvents include N-methyl-2-pyrrolidone, 2-pyrrolidone, ethanol, acetone, acetonitrile, methyl acetate, methylene chloride, ethyl acetate, methyl ethyl ketone, dimethylformamide, dimethyl sulfoxide, tetrahydrofuran, caprolactam, decylmethylsulfoxide, oleic acid, and 1-dodecylazacycloheptan-2-one. Preferably acetone or methylene chloride may be used. The amount of polymer dissolved in e.g. acetone or methylene chloride may be from about 10% w/v to about 40% w/v, preferably from about 15% w/v to about 30% w/v.

Optionally a an additive may be added to the polymer/solvent solution and/or-to the polyethylene glycol/drug substance solution. The additive may improve the solubility of the polymer and the drug substance of the active ingredient. The co-solvent may further modulate the drug release in vitro or in vivo. The additive may be present in a amount of from about 0.1% to about 20% w/v, preferably from about 1% to about 5%. Examples of such additives include methanol, ethanol, propylene glycol, liquid surfactant such as poly(oxyethylene) sorbitan esters (Tweens) or glycerin polyoxyethylene ester of castor oil (Cremophor EL), lactic acid, acetic acid, glycerol, N,N dimethylacetamide, benzyl benzoate, polyoxyethylated fatty acid, lecithin, soybean oil , seaflower oil, vegetable oils, cotton seed oils, oligormers of poly(l-lactide) of poly(d,l lactide) of poly(lactide co-glycolide) or a mixture of these oligomers.

The pharmaceutically active agent may be dissolved or dispersed in liquid polyethylene glycols (PEG), e.g. PEG 200, PEG 300, PEG 400, PEG 540 or PEG 600 (Handbook of Pharmaceutical Excipients loc. cit., p. 454) or PEG with modified end groups e.g. polyethylene glycol mono and di-alkyl ether (Handbook of Pharmaceutical Excipients loc. cit. p. 469) or polyethylenglycol 600 mono and di-acid at room temperature, e.g. 250° C., e.g. depending on its solubility in this solvent with or without a co-solvent.

Details of suitable excipients for use in the process of the invention are described in the “Handbook of Pharmaceutical Excipients”, Rowe, Sheskey and Weller, 4^{th} Edition 2003 which is incorporated by reference.

For the purpose of the invention “pharmaceutically active agent” means all substances that produce a pharmaceutical or a therapeutic effect. Examples of pharmaceutically active agents include but are not limited to peptides, polypeptides, proteins, carbohydrates, oligonucleotides, RNA and DNA. A few examples of peptides are antibodies, growth hormones, e.g. epidermal growth factor (EGF), prolactin, luliberin or luteinizing hormone releaseing hormone (LH-RH), glucagon, gastrin, pentagastrin, urogastron, secretin, enkephalins, endorphins, angiotensins, renin, bradykinin, bacitracins, polymyxins, colistins, tyrocidin, gramicidines, insulin, octreotide, e.g. as disclosed in U.S. Pat. No. 4,395,403, interferons, erythropoietin, calcitonin, heparin, somatostatin analogues, e.g. somatostatin pamoate or di-aspartate, cell stimulating factors and parathyroid hormones.

A preferred active agent may be a somatostatin analogue which is dissolved in polyethylene glycol. A more preferred active agent may be somatostatin pamoate or di-aspartate which may be dissolved 1:1 in polyethylene glycol to form a solution with up to 20 mg/ml of the active agent.

Somatostatin is a tetradecapeptide having the structure

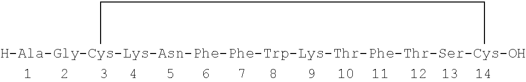

Somatostatin analogues of particular interest have been described e.g. in WO 97/01579 and WO 97/25977. Said somatostatin analogues comprise the amino acid sequence of formula I

−(D/L)Trp-Lys-X₁-X₂- I

wherein X₁ is a radical of formula (a) or (b)

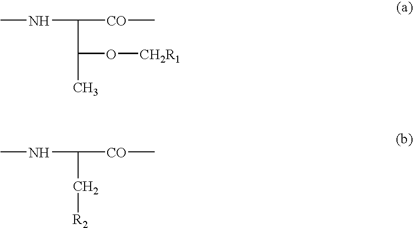

wherein R₁ is optionally substituted phenyl, wherein the substituent may be halogen, methyl, ethyl, methoxy or ethoxy,

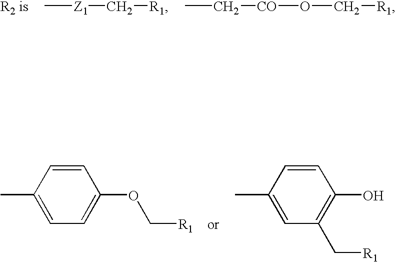

wherein Z₁ is O or S, and

X₂ is an α-amino acid having an aromatic residue on the C_{α}, side chain, or an amino acid unit selected from Dab, Dpr, Dpm, His,(Bzl)HyPro, thienyl-Ala, cyclohexyl-Ala and t-butyl-Ala, the residue Lys of said sequence corresponding to the residue Lys⁹ of the native somato-statin-14.

By somatostatin analogue as used herein is meant a straight-chain or cyclic peptide derived from that of the naturally occurring somatostatin-14, comprising the sequence of formula I and wherein additionally one or more amino acid units have been omitted and/or replaced by one or more other amino acid radical(s) and/or wherein one or more functional groups have been replaced by one or more other functional groups and/or one or more groups have been replaced by one or several other isosteric groups. In general the term covers all modified derivatives of the native somatostatin-14 comprising the above sequence of formula I which have binding affinity in the nM range to at least one somatostatin receptor subtype as defined hereinafter.

Preferably, the somatostatin analogue is a compound in which the residues at positions 8 through 11 of the somatostatin-14 are represented by the sequence of formula I as defined above.

More preferably, the somatostatin analogue is a compound as disclosed above comprising a hexapeptide unit, the residues at positions 3 through 6 of said hexapeptide unit comprising the sequence of formula 1. Particularly preferred is a somatostatin hexapeptide wherein the residues at positions 1 and 2 of the hexapeptide unit may be any of those as known in the art, e.g. as disclosed by A. S. Dutta in Small Peptides, Vol.19, 292-354, Elsevier, 1993, or as substituents for, Phe⁶ and/or Phe⁷ of somatostatin-14.

More particularly the somatostatin analogue is a compound in which the hexapeptide unit is cyclic, e.g. having a direct peptide linkage between the (x-carbonyl group of the residue at position 6 and the α-amino group of the residue at position 1.

While Lys, XI and X₂ in the sequence of formula I have the L-configuration, Trp may have the D- or L-configuration. Preferably Trp has the D-configuration.

X₁ is preferably a residue of formula (a) or (b), R₂ being preferably

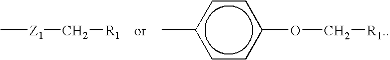

When X₂ comprises an aromatic residue on the C_{α}side chain, it may suitably be a natural or unnatural α-amino acid, e.g. Phe, Tyr, Trp, Nal, Pal, benzothienyl-Ala, Tic and thyronin, preferably Phe or Nal, more preferably Phe. X₂ is preferably an α-amino acid bearing an aromatic residue on the C_{α}side chain.

When R₁ is substituted phenyl, it may suitably be substituted by halogen, methyl, ethyl, methoxy or ethoxy e.g. in ortho and/or para. More preferably R₁ is unsubstituted phenyl.

Z₁ is preferably O.

Representative compounds of the invention are e.g. compounds of formula (II)

| cyclo[A-ZZₐ-(D/L)Trp-Lys-X₁-X₂] | (II) |
|---|---|
| 1 2 3 4 5 6 | |

wherein

X₁ and X₂ are as defined above,

A is a divalent residue selected from Pro,

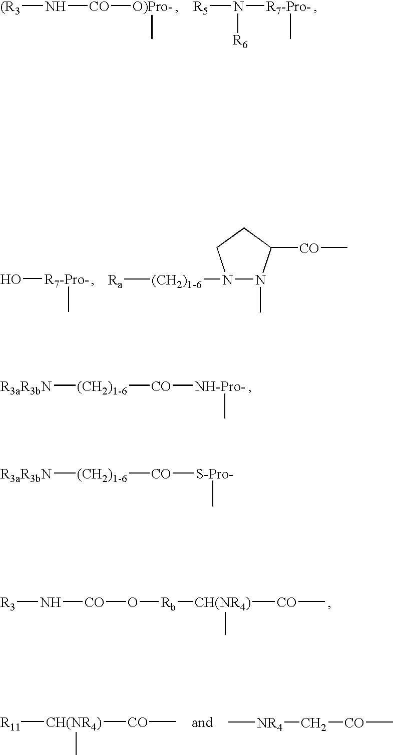

wherein R₃ is NR₈R₉—C₂-₆alkylene, guanidino-C₂-₆alkylene or C₂-₆alkylene-COOH, R₃ₐis H, C₁-₄alkyl or has independently one of the significances given for R₃, R_{3b}is H or C₁₋₄alkyl, Rₐ is OH or NR₅R₆, R_{b} is —(CH₂)₁₋₃— or —CH(CH₃)—, R₄ is H or CH₃, R₄ₐ is optionally ring-substituted benzyl, each of R₅ and R₆ independently is H, C₁₋₄alkyl, ω-amino-C₁₋₄alkylene, ω-hydroxy-C₁₋₄alkylene or acyl, R₇ is a direct bond or C₋₆alkylene, each of R8 and R₉ independently is H, C₁₋₄alkyl, ω-hydroxy-C₂₋₄alkylene, acyl or CH₂OH—(CHOH)_{c}—CH₂— wherein c is 0, 1, 2, 3 or 4, or R₈ and R₉ form together with the nitrogen atom to which they are attached a heterocyclic group which may comprise a further heteroatom, and R₁₁ is optionally ring-substituted benzyl, —(CH₂)₁₋₃—OH, CH₃—CH(OH)— or —(CH₂)₁₋₅—NR₅R₆, and

ZZₐ is a natural or unnatural α-amino acid unit.

ZZₐ may have the D- or L-configuration. When ZZₐ is a natural or unnatural α-amino acid unit, it may suitably be e.g. Thr, Ser, Ala, Val, lie, Leu, Nle, His, Arg, Lys, Nal, Pal, Tyr, Trp, optionally ring-substituted Phe or N^{α}-benzyl-Gly. When ZZₐ is Phe, the benzene ring thereof may be substituted by e.g. NH₂, NO₂, CH₃, OCH₃ or halogen, preferably in para position. When ZZₐ is Phe, the benzene ring thereof is preferably unsubstituted.

When A comprises a Pro amino acid residue, any substituent present on the proline ring, e.g. R₃—NH—CO—O— etc., is preferably in position 4. Such substituted proline residue may exist in the cis form, e.g.

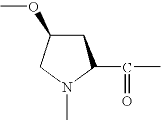

as well as in the trans form. Each geometric isomer individually as well as mixtures thereof are compounds of the invention.

When A is

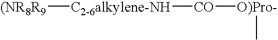

where NR₈R₉ forms a heterocyclic group, such group may be aromatic or saturated and may comprise one nitrogen or one nitrogen and a second heteroatom selected from nitrogen and oxygen. Preferably the heterocyclic group is e.g. pyridyl or morpholino. C₂₋₆Alkylene in this residue is preferably —CH₂—CH₂—.

Any acyl as R₅, R₆, R₈ and R₉ in A may be e.g. R₁₂CO— wherein R₁₂ is H, C₁₋₄alkyl, C₂₋₄alkenyl, C₃₋₆cycloalkyl or benzyl, preferably methyl or ethyl. When R₄ₐ or R₁₁ in A is ring-substituted benzyl, the benzene ring may be substituted as indicated above for ZZₐ.

Particularly preferred are compounds of formula III

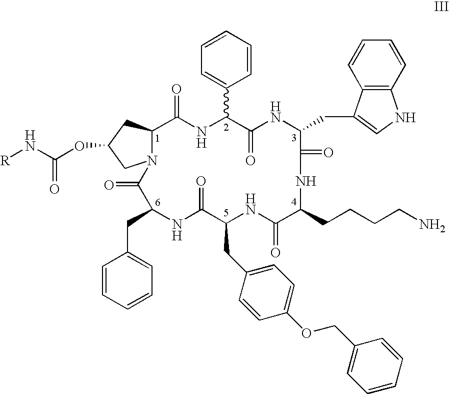

wherein the configuration at C-2 is (R) or (S) or a mixture thereof, and

wherein R is NR₁₀R₁-C₂₋₆alkylene or guanidine-C₂₋₆alkylene, and each of R₁₀ and R₁₁ independently is H or C₁₋₄alkyl,

in free form, in salt form or protected form.

Preferably R is NR₁₀R₁₁—C₂₋₆alkylene. Preferred compounds of formula II are the compounds wherein R is 2-amino-ethyl, namely cyclo[{4-(NH₂—C₂H₄—NH—CO—O—)Pro}-Phg-DTrp-Lys-Tyr(4-Bzl)-Phe] (referred herein to as Compound A) and cyclo[{4-(NH₂—C₂H₄—NH—CO—O—)Pro}-DPhg-DTrp-Lys-Tyr(4-Bzl)-Phe], in free form, salt form or protected form. Phg means —HN—CH(C₆H₅)—CO— and Bzl means benzyl.

A compound of the invention in protected form corresponds to a somatostatin analogue wherein at least one of the amino groups is protected and which by deprotection leads to a compound of formula II, preferably physiologically removable. Suitable amino protecting groups are e.g. as disclosed in “Protective Groups in Organic Synthesis”, T. W. Greene, J. Wiley & Sons NY (1981), 219-287, the contents of which being incorporated herein by reference. Example of such an amino protecting group is acetyl.

A compound of the invention may exist e.g. in free or salt form. Salts include acid addition salts with e.g. inorganic acids, polymeric acids or organic acids, for example with hydrochloric acid, acetic acid, lactic acid, aspartic acid, benzoic acid, succinic acid or pamoic acid. Acid addition salts may exist as mono- or divalent salts, e.g. depending whether 1 or 2 acid equivalents are added. Preferred salts are the lactate, aspartate, benzoate, succinate and pamoate including mono- and di-salts, more preferably the aspartate di-salt and the pamoate monosalt.

In another aspect the invention provides a pharmaceutical composition comprising a somatostatin analogue, e.g. somatostatin pamoate, obtainable by the process of the invention. The composition may further comprise a polymer and polyethylene glycol as described above. In a further aspect of the invention the composition obtainable by the process of the present invention may be in liquid form, e.g. a solution. After sterile filtration through a 0.2 micrometer filter the liquid composition, e.g. solution, may be placed in a syringe. Sterilization may also be achieved by terminal sterilization with gamma irradiation at 20 to 30 kGy preferably at 25 kGy under cooled conditions, e.g. 2 to 8° C. or −70° C. The sterilized solution may be injected through a needle, e.g. an up to 20 G needle, into the body subcutaneously or intramuscularly. Once in place the solvent, e.g. polyethylene glycol will dissipate and the polymer together with the pharmaceutically active agent solidifies to form the implant. Accordingly to the invention, preferably a prefilled syringe may be provided together with instructions for use.

In another aspect the invention provides a depot formulation for extended release of the pharmaceutically active agent. The implant formed after injection into the body may release the active agent over an extended period of time. The desired release profile may depend on the kind of monomer, whether a homo- or a co-polymer or whether a mixture of polymers is employed. The release period may range from 1 up to 12 weeks, e.g. 1 to 8 weeks.

The compositions of the invention are useful for treatment of the known indications of the particular active agent incorporated in the polymer. Compositions of the invention comprising a somatostatin anologue may be useful in the following indications:

a) for the prevention or treatment of disorders with an aetiology comprising or associated with excess GH-secretion and/or excess of IGF-1 e.g. in the treatment of acromegaly as well as in the treatment of type I or type II diabetes mellitus, especially complications thereof, e.g. angiopathy, diabetic proliferative retinopathy, diabetic macular edema, nephropathy, neuropathy and dawn phenomenon, and other metabolic disorders related to insulin or glucagon release, e.g. obesity, e.g. morbid obesity or hypothalamic or hyperinsulinemic obesity,

b) in the treatment of enterocutaneous and pancreaticocutaneous fistula, irritable bowel syndrome, inflammatory diseases, e.g. Grave's Disease, inflammatory bowel disease, psoriasis or rheumatoid arthritis, polycystic kidney disease, dumping syndrome, watery diarrhea syndrome, AIDS-related diarrhea, chemotherapy-induced diarrhea, acute or chronic pancreatitis and gastrointestinal hormone secreting tumors (e.g. GEP tumors, for example vipomas, glucagonomas, insulinomas, carcinoids and the like), lymphocyte malignancies, e.g. lymphomas or leukemias, hepatocellular carcinoma as well as gastrointestinal bleeding, e.g variceal oesophagial bleeding,

c) for the prevention or treatment of angiogenesis, inflammatory disorders as indicated above including inflammatory eye diseases, macular edema, e.g. cystoid macular edema, idiopathic cystoid macular edema, exudative age-related macular degeneration, choroidal neovascularization related disorders and proliferative retinopathy,

d) for preventing or combating graft vessel diseases, e.g. allo- or xenotransplant vasculo-pathies, e.g. graft vessel atherosclerosis, e.g. in a transplant of organ, e.g. heart, lung, combined heart-lung, liver, kidney or pancreatic transplants, or for preventing or treating vein graft stenosis, restenosis and/or vascular occlusion following vascular injury, e.g. caused by catherization procedures or vascular scraping procedures such as percutaneous transluminal angioplasty, laser treatment or other invasive procedures which disrupt the integrity of the vascular intima or endothelium,

e) for treating somatostatin receptor expressing or accumulating tumors such as pituitary tumors, e.g. Cushing's Disease, gastro-enteropancreatic, carcinoids, central nervous system, breast, prostatic (including advanced hormone-refractory prostate cancer), ovarian or colonic tumors, small cell lung cancer, malignant bowel obstruction, paragangliomas, kidney cancer, skin cancer, neuroblastomas, pheochromocytomas, medullary thyroid carcinomas, myelomas, lymphomas, Hodgkins and non-Hodgkins lymphomas, bone tumours and metastases thereof, as well as autoimmune or inflammatory disorders, e.g. rheumatoid arthritis, Graves disease or other inflammatory eye diseases.

Preferably, the compositions of the invention are useful in the treatment of acromegaly and cancer, e.g. Cushing's Disease.

The activity and the characteristics of the liquid compositions of the invention may be indicated in standard clinical or animal tests.

Appropriate dosage of the composition of the invention will of course vary, e.g. depending on the condition to be treated (for example the disease type of the nature of resistance), the drug used, the effect desired and the mode of administration. For compositions of the invention comprising a somatostatin analogue satisfactory results are obtained on administration, e.g. parenteral administration, at dosages on the order of from about 0.2 to about 60 mg, preferably from about 5 to about 40 mg per injection per month or about 0.03 to about 1.2 mg per kg animal body weight per month, administered once or in divided doses. Suitable monthly dosages for patients are thus in the order of about 0.3 mg to about 40 mg of a somatostatin analogue, e.g. Compound A pamoate. The composition may be administered every 2 to 3 months. Suitable dosages for every 3 months administration are about 1 mg to about 180 mg.

Following is a description by way of example only of processes and compositions of the invention.

### EXAMPLE 1

The solubility of different polymers (linear polymer): in polyethylene glycol is tested. The solubility of the linear polymers (Resomer®) and the star polymer (Poly(D),L-lactide-co-glycolide), D,L PLG-Glu is shown in table 1.

TABLE 1

| | Polymer | | | |
|---|---|---|---|---|
| | Resomer ® | Resomer ® | | |
| | RG 50:50 | RG 502H | Resomer ® | D,L-PLG-Glu |
| | Mw: 2100 g/mol | Mw: 12000 g/mol | RG 502H | Mw: 50800 g/mol |
| Polymer | 10% | 10% | 20% | 10% |
| Concentration | | | | |
| In PEG 300 | | | | |
| Appearance | Clear solution | Clear solution | Clear solution | Clear solution |

### EXAMPLE 2

Preparation of 20 ml polymer/PEG solution:

4.004 g Resomer RG 502 H are dissolved in 13.3 ml acetone. 20 ml polyethylene glycol PEG is added to this solution together with 25 mg/ml of Compound A pamoate. The complete solution is stirred 4 hours at room temperature and N₂ urging under reduced pressure. After sterile filtration the solution is filled in a syringe. The obtained prefilled syringe may be used for subcutaneous administration.

### EXAMPLE 3

4 g of resomer RG502 H were dissolved in 6.6 ml methylene chloride. 0.250 g Compound A di-aspartate were dissolved in 2 ml water and added to 20 ml PEG300. Both polymer and drug substance solutions were mixed together. The methylene chloride was evaporated for 5 hours at 40° C. in a water batch resulting in a injectable in situ forming depot formulation of 1.25% w/v Compound A di-aspartate and 20% w/v Resomer RG502H in PEG 300.

### EXAMPLE 4

1.5 g of resomer RG502H and 1.0 g of oligomers of poly(lactide-co-glycolide) 50:50 were dissolved in 3.3 ml methylene chloride. 0.250 g Compound A-pamoate were dissolved in 10 ml PEG300. Both polymer and drug substance solutions were mixed together. The methylene chloride was evaporated for 5 hours at 40 ° C. in a water batch resulting in a injectable in situ forming depot formulation of 2.5% w/v Compound A pamoate and 15% w/v Resomer RG502H and 10% oligomers in PEG 300.

### EXAMPLE 5

1.54 g of resomer RG502H and 0.5 g of benzyl benzoate were dissolved in 3.3 ml methylene chloride. 0.250 g Compound A-pamoate were dissolved in 10 ml PEG300. Both polymer and drug substance solutions were mixed together. The methylene chloride was evaporated for 5 hours at 40° C. in a water batch resulting in a injectable in situ forming depot formulation of 2.5% w/v Compound A pamoate and 15% w/v Resomer RG502H and 5% benzyl benzoate in PEG 300.

### EXAMPLE 6

2.0 g of resomer RG502H were dissolved in 3.3 ml methylene chloride. 0.250 g Compound A-pamoate were dissolved in 10 ml PEG250 diethylether. Both polymer and drug substance solutions were mixed together. The methylene chloride was evaporated for 5 hours at 40° C. in a water batch resulting in a injectable in situ forming depot formulation of 2.5% w/v Compound A pamoate and 20% w/v Resomer RG502H in PEG 250 diethylether.

### EXAMPLE 7

4 g of resomer RG502H were dissolved in 6.6 ml methylene chloride. 0.50 g Compound A pamoate were dissolved in 20 ml PEG300. Both polymer and drug substance solutions were mixed together. The methylene chloride was evaporated for 5 hours at 40° C. in a water batch resulting in a injectable in situ forming depot formulation of 2.5% w/v Compound A pamoate and 20% w/v Resomer RG502H in PEG 300.

1: A liquid pharmaceutical composition comprising
(a) a biodegradable polymer;
(b) a polyethylene glycol having a molecular weight of less than 600 Daltons,
(c) a pharmaceutically active agent, and
(d) less than about 0.5% of a pharmaceutically acceptable organic solvent, and optionally
(e) an additive.

2: The liquid pharmaceutical composition according to claim 1 wherein the polyethylene glycol is PEG 200, PEG 300, PEG 400 or PEG 600 or di-alkyl ether PEG.

3: The liquid pharmaceutical composition according to claim 1 wherein the polymer is a linear or branched polylactide-co-glycolide.

4: The liquid pharmaceutical composition according to claim 1 wherein the pharmaceutically active agent is selected from the group consisting of peptides, polypeptides, proteins, carbohydrates, oligonucleotides, RNA and DNA.

5: The liquid pharmaceutical composition according to claim 1 wherein the pharmaceutically active agent is a somatostatin analogue.

6: The liquid pharmaceutical composition according to claim 1 wherein the pharmaceutically active agent is a cyclo[{4-(NH₂—C₂H₄—NH—CO—O)Pro}-Phg-DTrp-Lys-Tyr(4-Bzl)-Phe] pamoate or di-aspartate.

7: The liquid pharmaceutical composition according to claim 1 wherein the pharmaceutically acceptable organic solvent is chosen from the group consisting of N-methyl-2-pyrrolidone, 2-pyrrolidone, ethanol, acetone, acetonitrile, methyl acetate, ethyl acetate, methyl ethyl ketone, methylene chloride, dimethylformamide, dimethyl sulfoxide, tetrahydrofuran, caprolactam, decylmehylsulfoxide, oleic acid, and 1-dodecylazacycloheptan-2-one.

8: The liquid pharmaceutical composition according to claim 1 wherein the additive is chosen from methanol, ethanol, propylene glycol; a liquid surfactant or a glycerin polyoxyethylene ester of ricin oil, lactic acid, acetic acid, glycerol, N,N dimethylacetamide, benzyl benzoate, polyoxyethylated fatty acid, lecithin, soybean oil, seaflower oil, vegetable oils, cotton seed oils, oligomers of poly(l-lactide) of poly(d,l lactide) of poly(lactide co-glycolide) or a mixture of these oligomers.

9: A process for preparing a depot formulation comprising the steps:
i) dissolving a biodegradable polymer in a pharmaceutically acceptable organic solvent
ii) optionally adding an additive to the polymer/solvent solution of i)
iii) dissolving a pharmaceutically active agent in polyethylene glycol having a molecular weight of less than 600 Daltons
iv) mixing solution (i) and (iii) or (ii) and (iii)
v) removing the pharmaceutically acceptable organic solvent from the mixture.

10: The process according to claim 9 wherein optionally an additive is added to the polyethylene glycol before dissolving the pharmaceutically active agent in this solution.

11: The process according to claim 10 wherein the polymer is a linear or branched polylactide-co-glycolide.

12: The process according to claim 9 wherein at least two different polymers are dissolved in a pharmaceutically acceptable organic solvent.

13: The process according to claim 9 wherein the polyethylene glycol is PEG 200, PEG 300, PEG 400 or PEG 600 or di-alkyl ether PEG.

14: The process according to claim 9 wherein the pharmaceutically active agent is selected from the group consisting of peptides, polypeptides, proteins, carbohydrates, oligonucleotides, RNA and DNA.

15: The process according to claim 9 wherein the pharmaceutically active agent is a somatostatin analogue.

16: The process according to claim 9 wherein the pharmaceutically active agent is a pamoate or di-aspartate.

17: The process according to claim 9 wherein the pharmaceutically acceptable organic solvent is selected from the group consisting of N-methyl-2-pyrrolidone, 2-pyrrolidone, ethanol, acetone, acetonitrile, methyl acetate, ethyl acetate, methyl ethyl ketone, methylen chloride, dimethylformamide, dimethyl sulfoxide, tetrahydrofuran, caprolactam, decylmehylsulfoxide, oleic acid, and 1-dodecylazacycloheptan-2-one.

18: A pharmaceutical composition comprising a pharmaceutically active agent wherein the composition is obtainable by the process according to claim 9.

19: The pharmaceutical composition according to claim 18 wherein the pharmaceutically active agent is a somatostatin analogue.

20: The pharmaceutical composition according to claim 19 wherein the somatostatin analogue is a pamoate or a di-aspartate.

21: The pharmaceutical composition according to claim 1 for injection.

22: The pharmaceutical composition according to claim 1 which forms a solidified implant in the body after injection.

23: The pharmaceutical composition according to claim 1 wherein the active agent is released over 1 up to 12 weeks.

24: A prefilled syringe comprising the composition of claim 1 and instructions to use.
